(19) United States
(12) Patent Application Publication
(10) Pub. No.: US 2024/0090655 A1
(43) Pub. Date: Mar. 21, 2024

(54) Teeth Cleaning Systems with Integrated Sensors for Reliable Detection of Analytes in Saliva

(71) Applicant: Feno Labs, Inc., Culver City, CA (US)

(72) Inventors: Kenneth R Brown, Rancho Palos Verde, CA (US); Andreas Singh Dierks, Copenhagen (DK); Daniel Stage Kristoffersen, Copenhagen (DK); Hans L Michaelsen, Copenhagen (DK)

(73) Assignee: Feno Labs, Inc., Culver City, CA (US)

(21) Appl. No.: 18/470,631

(22) Filed: Sep. 20, 2023

Related U.S. Application Data
(60) Provisional application No. 63/376,440, filed on Sep. 21, 2022.

(51) Int. Cl. A46B 9/04; A61B 5/00; A61B 5/145

(52) U.S. Cl. CPC A46B 9/045; A61B 5/14546; A61B 5/682; A61B 5/683; A46B 2200/1066

(57) ABSTRACT

A teeth cleaning system that measures and analyzes analytes in a user's saliva. The system includes an actuated tray comprising brushing elements. At least one integrated sensor may be housed in a tray of said teeth cleaning system that can detect one or more analytes within said user's saliva. In operation, said integrated sensor is exposed to said user's saliva for measuring a concentration of said analyte in said user's saliva. An electrochemical analyzer and/or a processor may also be housed in a handle assembly for analyzing a signal related to at least one measuring of said concentration of said analyte. Said concentration readings and analysis may be sent to an external network, including a network that interfaces a mobile app for further analysis, presentation, or personalized advice and products sent to said user through said mobile app.

### PRIORITY CLAIM

This application claims priority to and claims benefit under 35 U.S.C. § 119(e) of U.S. Provisional Application having Ser. No. 63/376,440 filed Sep. 21, 2022. This Provisional patent application is hereby incorporated by reference herein in its entirety.

### FIELD

Various embodiments concern electric toothbrushes and, more specifically, electric toothbrushes with integrated sensors that monitor for the presence or concentration of analytes in saliva.

### BACKGROUND

The term “toothbrush” is commonly used to refer to an oral hygiene implement that can be used to clean the teeth, gums, and tongue. A toothbrush includes a head of tightly clustered bristles, atop of which toothpaste or another dentifrice can be applied, mounted on a handle that facilitates the cleaning of hard-to-reach areas of the mouth. At a high level, the toothpaste acts as an abrasive that aids in removing dental plaque and food from the teeth, and therefore running the bristles—with the toothpaste—across the teeth can help maintain the aesthetics and health of the teeth and oral cavity. However, toothbrushes are commonly used in conjunction with another implement to clean between the teeth where the bristles cannot reach, or cannot regularly reach, such as dental floss, tape, or interdental brushes.

To clean the teeth, an individual can grasp the handle and then run the bristles across the surface of the teeth. There are several downsides to manual operation of toothbrushes, however. First, maintaining proper alignment of the bristles with respect to the teeth can be difficult. For example, in order to properly brush with a straight-bristled toothbrush, the individual should not scrub horizontally over the necks of the teeth, nor should she press the bristles too hard against the teeth. Second, even with proper alignment, some teeth may be difficult to clean consistently. For example, individuals often find it difficult to consistently brush the molars and lingual sides of other teeth, and therefore dental plaque tends to build up over time. Not only is it difficult to apply appropriate amounts of force to these areas, but some individuals struggle to even contact these areas with the bristles.

Electronic toothbrushes (also called “powered toothbrushes”) were developed in an effort to address some of the downsides of “manual toothbrushes.” The terms “electric toothbrush” and “powered toothbrush” are commonly used to refer to a toothbrush with bristles that make rapid, automatic motions—for example, lateral oscillation where a bristle head moves back and forth or rotational oscillation where a bristle head rotates clockwise, counterclockwise, or alternatives between clockwise and counterclockwise—in order to clean the teeth. The mechanism used to achieve motion of the bristles may depend on the desired speed of the oscillation. Motion at sonic speeds may be induced or caused by a motor housed in the handle, while motion at ultrasonic speeds may be induced or caused by a piezoelectric crystal. Regardless of its form, the mechanism is electrically coupled to a power source that is rechargeable between uses.

### SUMMARY

In one aspect of the disclosure, a teeth cleaning system is disclosed wherein a handle assembly is removably connected to a tray that is configured to be inserted into a user's mouth to clean said user's teeth. When said tray is inserted into said user's mouth, it surrounds said upper and lower teeth of said user for cleaning all of said user's teeth. In some embodiments, said tray has one or more apertures for housing at least one integrated sensor that is configured to identify a concentration of at least one analyte in said user's saliva. An electrochemical analyzer may be used to receive a reading from the at least one integrated sensor. Electrochemical analyzers may be components for obtaining, determining, and analyzing chemical measurements from said integrated sensors. The teeth cleaning system may also include a transmitter for transmission of at least one signal that represents said concentration of at least one analyte in said user's saliva to an external network. In some embodiments, said external network is in communication with an app on a user's mobile device, wherein said teeth cleaning device can send and receive data from said app on a user's mobile device.

In another aspect of the disclosure, a teeth cleaning system is disclosed that may include one or more integrated sensors disposed on or adjacent to an inner wall of said tray for exposure to said user's saliva. In comparison to traditional toothbrushes, the tray may expose the integrated sensors to additional saliva for more accurate readings and assist in avoiding toothpaste interference. In some embodiments, a first integrated sensor may be configured to identify a first concentration of a first analyte in a user's saliva and a second integrated sensor may be configured to identify a second concentration of a second analyte.

Further, a teeth cleaning system is disclosed which may communicate with said user through an external network and an app on said user's mobile device. In some embodiments, the app may report concentrations of said analyte and provide analysis of said concentrations. Thus, the app not only receives information from said teeth cleaning system, but may communicate data to said teeth cleaning system. For example, said app may inform said user that said integrated sensors and/or said tray need to be replaced.

It is understood that other configurations of the subject technology will become readily apparent to those skilled in the art from the following detailed description, wherein various configurations of the subject technology are shown and described by way of illustration.

As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

FIG. 1 illustrates an example of a teeth cleaning system (or simply “system”) that includes a tray assembly (or simply “tray”) and a handle assembly (or simply “handle”).

FIG. 2 is a perspective view of an example of a teeth cleaning system.

FIG. 3 is another perspective view of the teeth cleaning system shown in FIG. 2.

FIG. 4 is a perspective view of a motorized handle that may be part of the teeth cleaning system of FIG. 1.

FIG. 5 is a perspective, internal view of the motorized elements in the handle of FIG. 4.

FIG. 6 illustrates an example of a teeth cleaning system that is able to implement a computer program—called an “analysis platform”—designed to parse signals generated by integrated sensors of the teeth cleaning system.

FIG. 7 illustrates a network environment that includes an analysis platform that is executed by a teeth cleaning system.

Embodiments are illustrated by way of example and not limitation in the drawings. While the drawings depict various embodiments for the purpose of illustration, those skilled in the art will recognize that alternative embodiments may be employed without departing from the principles of the technology. Accordingly, while specific embodiments are shown in the drawings, the technology is amenable to various modifications.

### DETAILED DESCRIPTION

Various diseases can impact—either directly or indirectly—the oral health of affected individuals. For example, lack of ascorbic acid (also called “vitamin C”) can lead to a condition called “scurvy” that commonly causes muscle weakness, swollen or bleeding gums, loss of teeth, tiredness, and depression. While these symptoms may require diagnosis by a healthcare professional, the underlying issue—namely, lack of vitamin C—is more readily detectable. As another example, lack of sodium can lead to a condition called “hyponatremia” that commonly causes nausea, vomiting, weakness, and generally confusion. Again, while these symptoms may require diagnosis by a healthcare professional, the underlying issue—namely, lack of sodium—is more readily detectable.

A core problem with detecting these diseases, especially in the early stages when treatment is most effective, is the lack of symptoms. Simply put, an individual is unlikely to visit a healthcare facility for diagnosis by a healthcare professional until her symptoms have progressed. So while the underlying issue may be readily detectable, diagnosing these diseases remains difficult. While powered toothbrushes generally lead to meaningful improvement in oral health, powered toothbrushes are unable to address the underlying issues of these diseases, as these diseases are not resolvable by improving oral health. However, the consistent, periodic use of powered toothbrushes makes them a solid option for monitoring oral health in such a manner that these diseases can be more quickly detected and precisely monitored.

Introduced here, therefore, are teeth cleaning systems with integrated sensors that monitor for the presence or concentration of analytes in saliva. Because the integrated sensors are designed and then implemented to examine the saliva, the term “integrated sensor” may be used synonymously with “salivary sensor.” Each integrated sensor may be incorporated into a teeth cleaning system in such a manner that the saliva can be tested during normal use. For example, each integrated sensor may be incorporated in the teeth cleaning system such that, in operation, its sensing surface is situated along the surface of the teeth cleaning system, and therefore exposed to the saliva inside the mouth.

In operation, each integrated sensor can produce, as output, a signal that is indicative of the presence or concentration of analyte molecules (or simply “analyte”). Generally, the signal is representative of a sequence of values in temporal order that indicate the presence or concentration of the analyte at corresponding points in time. The term “analyte” may be used to refer to any substance, biological constituent, or chemical constituent that is of interest in an analytical procedure. Examples of analytes include chemical elements such as sodium, potassium, calcium, magnesium, bicarbonates, and phosphates; vitamins such as vitamins A and E, vitamin Bl, vitamin B2, vitamin B3, vitamin B6, and vitamin C; glucose; immunoglobulins; proteins; enzymes; nitrogenous products such as urea and ammonia; hormones such as testosterone, cortisol, estrogen, progesterone, androgen; bacteria (e.g., in terms of bacterial load); inflammatory markers such as cytokines, tumor necrosis factor, and acute phase proteins; and tumor markers such as certain transcriptomes and microbiota.

In some embodiments, the signal is processed and then analyzed by the teeth cleaning system. For example, proprietary machine learning could be employed “in device” such that the health of a user is locally analyzed without requiring that the signal be transmitted external to the teeth cleaning system. In such embodiments, analyses of the signal may be transmitted external to the teeth cleaning system, for example, to a computer program executing on an electronic device that is communicatively connectable to the teeth cleaning system. Through the computer program, a user may be able to review the analyses. Note that the “user” of the computer program could be the person whose teeth are brushed by the teeth cleaning system or some other person.

In other embodiments, the signal is processed and/or analyzed external to the teeth cleaning system. For example, the signal may be forwarded—in its raw form or processed form—to an electronic device for analysis by a computer program executing thereon. The signal could be streamed to the electronic device in near real time as its values are generated, or the signal could be transmitted to the electronic device such that values are received in “batches.” For example, the values may be forwarded by the teeth cleaning system to the electronic device when brushing ends, when the teeth cleaning system is recharged, when a predetermined time (e.g., 12 AM) has passed, or when a predetermined duration (e.g., 24 hours) has elapsed. Accordingly, values could be provided to the electronic device for analysis by the computer program in a continual, periodic, or ad hoc manner.

One of the long-term goals of integrating sensors into teeth cleaning systems is to utilize the integrated sensors to provide insight into the health of users. For example, these insights into the health of users may allow for improved understanding of nutrition, wellness, and the like, that can be surfaced through a computer program (e.g., in the form of a mobile application executing on a mobile phone or tablet computer). Moreover, these insights into the health of users may allow for more targeted treatment, for example, through the generation of personalized recommendations or products. A recommendation may request that a user take an action to address an underlying issue (e.g., consume foods high in vitamin C to address a deficiency), or a recommendation may request that the user seek further treatment (e.g., by indicating that the user should contact a healthcare professional). If you are low on magnesium or vitamin C, magnesium or vitamin C could be added to the user's toothpaste and shipped to the user or corresponding supplements could be shipped to the user.

Note that embodiments may be described with reference to sensors located in certain positions or able to detect certain analytes for the purpose of illustration. Those skilled in the art will recognize that the teeth cleaning systems described herein are amenable to change based on, for example, the analyte of interest. Similarly, embodiments may be described with reference to a teeth cleaning system that, in operation, is designed to fit inside of the oral cavity similar to a mouthpiece. However, sensors could be integrated within a powered toothbrush having a more traditional form.

Brief definitions of some terms used throughout the Detailed Description are provided below. References in this description to “an embodiment” or “some embodiments” means that the feature being described is included in at least one embodiment of the technology. Occurrences of such phrases do not necessarily refer to the same embodiment, nor are they necessarily referring to alternative embodiments that are mutually exclusive of one another. The terms “comprise,” “comprising,” and “comprised of” are to be construed in an inclusive sense rather than an exclusive or exhaustive sense (i.e., in the sense of “including but not limited to”). The term “based on” is also to be construed in an inclusive sense rather than an exclusive or exhaustive sense. Accordingly, the term “based on” is intended to mean “based at least in part on” unless otherwise noted. The terms “connected,” “coupled,” and variants thereof are intended to include any connection or coupling between two or more elements, either direct or indirect. The connection or coupling can be physical, electrical, or a combination thereof. For example, elements may be electrically coupled to one another despite not sharing a physical connection.

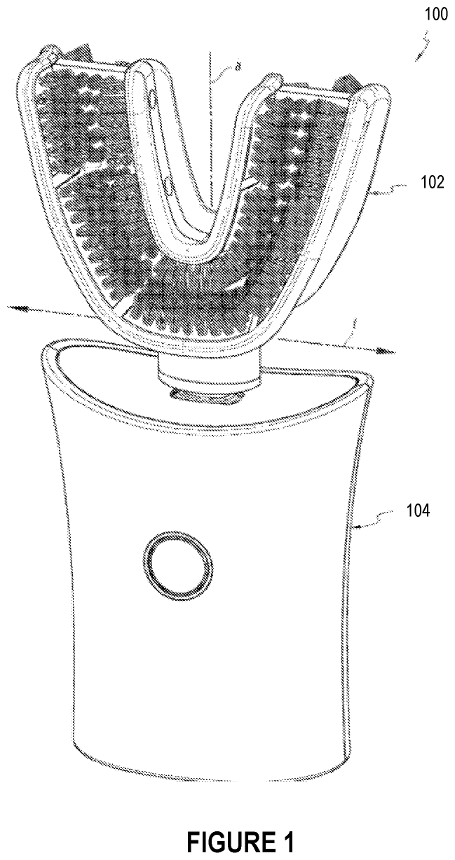

FIG. 1 illustrates an example of a teeth cleaning system 100 (or simply “system”) that includes a tray assembly 102 (or simply “tray”) and a handle assembly 104 (or simply “handle”). The tray 102 can be connected to the handle 104, such that the tray 102 is actuated to brush the teeth of a user. Generally, the tray 102 is designed to be inserted in the mouth axially along an axis “a.” The a-axis is representative of a line or plane that extends from the bottom of the handle 104 to the front of the tray 102 and is generally the direction of insertion. When the handle 104 is operated, a motor element (not shown) may actuate the tray 102 laterally along an axis “i.” For example, the left and right sides of the tray 102 may move toward the left and right sides of the handle 104. In some embodiments, the motor element is further able to actuate the tray longitudinally along the a-axis.

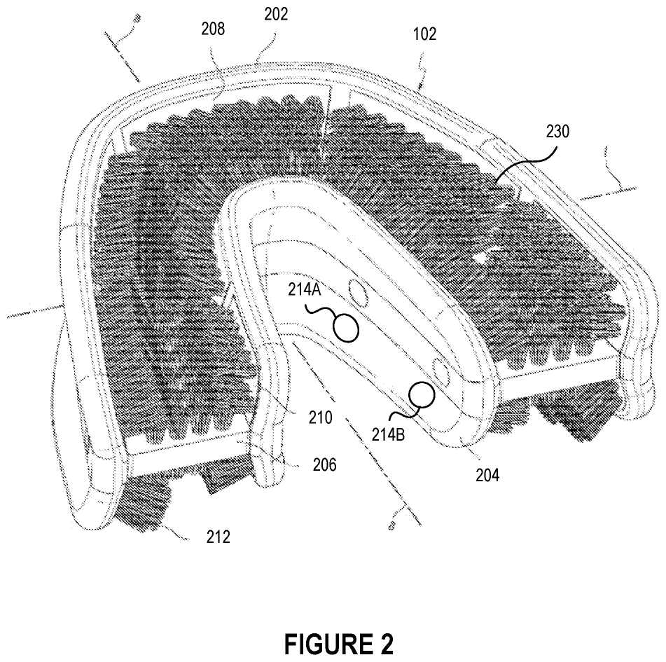

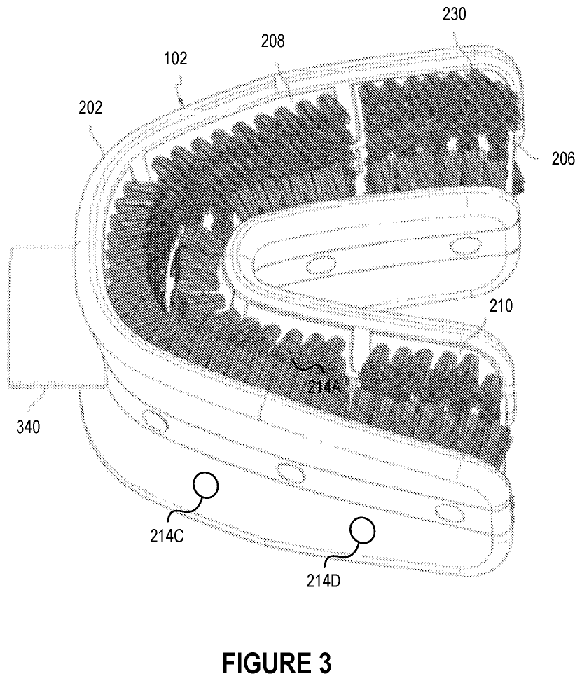

Referring to FIGS. 2-3, the tray 102 may be generally arcuate to loosely index to the shape of the jaw. The tray 102 may include a structural body that has an outer arcuate wall 202 and an inner arcuate wall 204. In some embodiments, a baffle 206 is positioned to transversely span across an inner surface 208 of the outer arcuate wall 202 and an inner surface 210 of the inner arcuate wall 204. The baffle 206 may follow the general arcuate shape of the tray 102. In some embodiments, the baffle 206 is parallel to the plane of the i-axis and transverse to the a-axis as shown in FIG. 2. The arrangement of the baffle 206 between the outer arcuate wall 202 and inner arcuate wall 204 may define an upper arcuate channel for receiving the teeth of the upper jaw and a lower arcuate channel for receiving the teeth of the lower jaw. Brushing elements 212 can be positioned in the channels of the tray 102. While the brushing elements 212 may be referred to as “bristles,” “bristle hairs,” or “strands.” The bristles could be made of nylon, microfiber, sponge (e.g., organic sponge or non-organic sponge), fabric, rubber, silicone, bamboo, or another firm yet flexible material.

The outer arcuate wall 202, inner arcuate wall 204, and top and bottom surfaces of the baffle 206 may include receptacles (also called “holes”) for retaining bristles 230. Each hole may be designed to hold a bundle of bristles. The free ends of the bristles may be arranged to contact the anterior, posterior, or top surfaces of each tooth, as well as reach between the teeth and gums. In some embodiments, the bristles projecting from the inner surface 208 of the outer arcuate wall 202 and the inner surface 210 of the inner arcuate wall 204 project at an acute angle relative to the plane defined along the upper or lower surface of the baffle 206. As the tray 102 is actuated in operation, the bristles on the baffle 206 generally brush the top surfaces of the teeth. Bristles on the inner surface 208 of the outer arcuate wall 202 and the inner surface 210 of the inner arcuate wall 204 generally brush the anterior and posterior surfaces of the teeth. The angle of the bristles on the inner surface 208 of the outer arcuate wall 202 and the inner surface 210 of the inner arcuate wall 204 may promote brushing along the height of the anterior or posterior face of each tooth. Note that the bristles along the innermost and outermost rows of the baffle 206 may partially overlap with the bristles along the inner surface 208 of the outer arcuate wall 202 and the inner surface 210 of the inner arcuate wall 204 that are closest to the baffle 206. Further information regarding the system 100 can be found in US Publication No. 2021/0161284, titled “Teeth Cleaning System and Method of Use” and is incorporated by reference herein in its entirety.

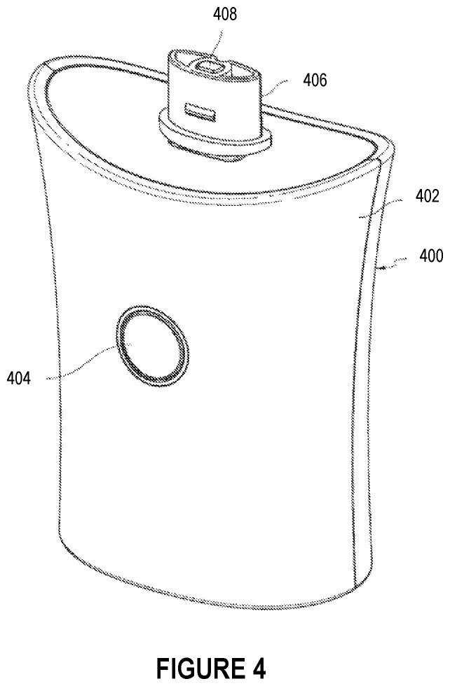

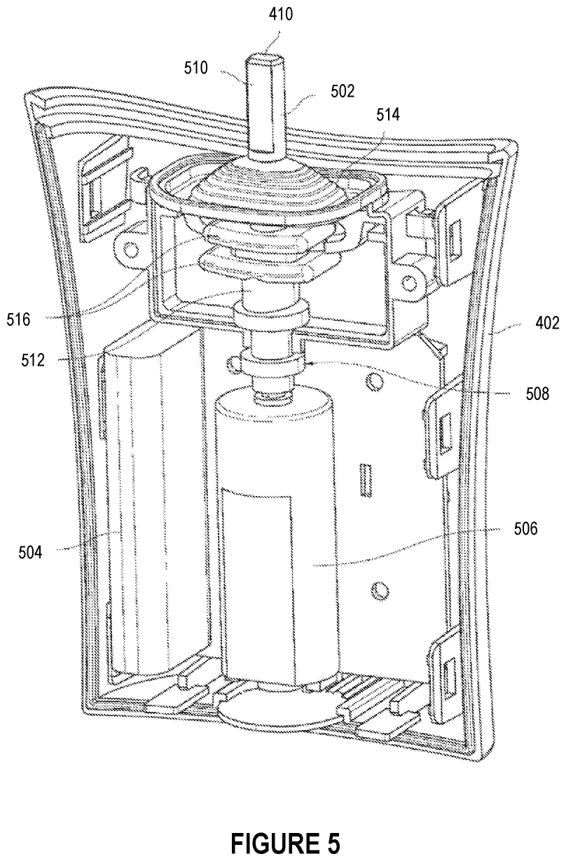

As shown in FIG. 3, the tray 102 may include a connector 340 that is coupled to the outer surface of the outer arcuate wall 202. The connector 340 may be configured to receive the oscillator mechanism that is discussed below in greater detail. For example, some embodiments may include a mating element (e.g., a “female” receptacle) that can receive a drive element, as discussed below with respect to FIG. 4-5, that actuates the tray 102.

Powered toothbrushes like the teeth cleaning system 100 generally have an undeniable impact on oral health. Research has shown that powered toothbrushes not only remove more plaque than manual toothbrushes, but also reduce gingival inflammation more than manual toothbrushes. While these improvements in oral health should not be discounted, some individuals who use powered toothbrushes will inevitably suffer from underlying diseases that affect oral health by affecting the salivary composition. While powered toothbrushes may not be able to address the underlying diseases, their consistent use makes them a solid option for monitoring the salivary composition in such a manner that the underlying diseases can be more quickly detected and precisely monitored.

To monitor the salivary composition, one or more sensors can be integrated into the teeth cleaning system 100. Specifically, one or more sensors can be integrated into the tray 102, and each integrated sensor can produce, as output, a signal that is representative of value indicative of the presence or concentration of a corresponding analyte in the saliva. The tray 102 can include integrated sensors 214A-B that are arranged along the inner arcuate wall 204 as shown in FIG. 2. Note that integrated sensors could be situated along the outer arcuate wall 202 instead of, or in addition to, the inner arcuate wall 204. In FIG. 3, for example, integrated sensors 214C-D are arranged along the outer arcuate wall 202. Integrated sensors that are located along the inner arcuate wall 204 are generally exposed to comparatively large amounts of saliva, though there is also a moderate chance of exposure to, or contact by, the tongue, which may result in degradation. Integrated sensors that are located along the outer arcuate wall 202 are less likely to be contacted by the tongue, as these sensors will be located proximate to the inner surface of the cheek. However, integrated sensors located along the outer arcuate wall 202 are generally exposed to less saliva than integrated sensors along the inner arcuate wall 204.

Whether a given integrated sensor is located along the outer arcuate wall 202 or inner arcuate wall 204 may depend on the amount of saliva that is needed or preferred to test for the corresponding analyte. In some embodiments, the use life of each integrated sensor 214A-D may be matched with the use life of said tray 102 such that the integrated sensors 214A-D are replaced when said tray 102 is replaced. In general, a mouthpiece or tray 102 may be able to capture saliva better than other electronic toothbrushes, as the saliva may gather in the tray 102 and there may be less interference from toothpaste.

In addition to the integrated sensors 214A-D being able to be placed in multiple positions on the tray 102, the integrated sensors 214A-D may be removed and replaced in those locations in the tray 102. As discussed above, degradation may occur and said integrated sensors 214A-D may need to be replaced to function as desired. Thus, the integrated sensors 214A-D may be secured in their position through form fitting where the integrated sensor 214A-D shape and size match the aperture in the tray 102, thus creating a snug fit where the integrated sensor 214A-D remains in place during operation but can be removed by the user. In FIGS. 2-3, a cylinder shape integrated sensor 214A-D may fit into a circular aperture in the tray 102. Integrated sensors 214A-D may also comprise different shapes that could allow the integrated sensors 214A-D to slide in and out of said apertures. For example, the integrated sensors 214A-D may have a rectangular shape that would allow insertion and extraction through a rectangular aperture in the tray 102. Complementary rails or protrusions on said tray 102 and said integrated sensors 214A-D may also allow said integrated sensors 214A-D to be inserted and extracted from said tray 102. During operation, there should be a tight fit for the integrated sensor 214A-D in the tray 102, and in most embodiments the integrated sensors 214A-D should touch or rest against one or more portions of said tray 102 to prevent movement or release. Reminders to replace said tray 102 and/or said integrated sensors 214A-D may be sent to said teeth cleaning system for display to said user or may be displayed to said user through an app on said user's mobile device. A light or display (not shown) on said handle assembly 104 could indicate that replacement of said tray 102 and/or said integrated sensors 214A-D is required.

In operation, the integrated sensors 214A-B can produce, as output, signals that are indicative of the presence or concentration of corresponding analytes. Generally, the signal is representative of a sequence of values in temporal order that indicate the presence or concentration of the analyte at corresponding points in time. Examples of analytes include chemical elements such as sodium, potassium, calcium, magnesium, bicarbonates, and phosphates; vitamins such as vitamins A and E, vitamin Bl, vitamin B2, vitamin B3, vitamin B6, and vitamin C; glucose; immunoglobulins; proteins; enzymes; nitrogenous products such as urea and ammonia; hormones such as testosterone, cortisol, estrogen, progesterone, androgen; bacteria (e.g., in terms of bacterial load); inflammatory markers such as cytokines, tumor necrosis factor, and acute phase proteins; and tumor markers such as certain transcriptomes and microbiota. Each sensor may track and identify the presence or concentration of one or more of said values of analytes. Said integrated sensors 214A-B may be connected to electrochemical analyzers (not shown) in said teeth cleaning system 100 by conductive traces. Electrochemical analyzers may be components for obtaining, determining, and analyzing chemical measurements from said integrated sensors. Data from these electrochemical analyzers could be connected to or transmit data to processors for further analysis before external transmission. For example, said handle assembly 104 may house said electrochemical analyzers and/or said processors for analysis of readings from said integrated sensors. 214A-B. The integrated sensors 214A-B may also produce output signals related to said degradation of said integrated sensors 214A-B, which could indicate that said user needs to replace said integrated sensors 214A-B.

FIG. 4 is a perspective view of a motorized handle 400 that may be part of the teeth cleaning system 100 of FIG. 1. The handle 400 includes a casing 402 and a button 404 that when triggered, activates the actuation of the tray 102. The handle 400 may house an oscillator assembly as discussed below with reference to FIG. 5. The oscillator assembly can include a shaft that protrudes from the top end of the casing 402. The shaft may be covered by a sleeve 406. The sleeve 406 may be a male connection that is configured to slide into the opening in the connector 340. A distal end 408 of the shaft may be configured to mate with a mating element in the connector 340. The oscillator assembly may be configured to move the shaft from side to side. When the shaft is actuated, the tray 102 is driven to oscillate from one side to the other side in a lateral direction.

While the above mating configuration was described with the sleeve 406 attached to the handle 400, it should be understood that in other embodiments, the sleeve 406 may instead be in the connector 340 and the distal end 408 of the shaft may be inserted through the interior of the sleeve to connect to the mating element.

FIG. 5 is a perspective, internal view of the motorized elements in the handle 400 of FIG. 4. The oscillator assembly 502 and other driving elements inside the casing 402 are shown according to an exemplary environment. The actuation from the handle 400 may be driven by a power source 504 (192), which may be a battery or a storage cell connected to a wall outlet source for “plugged-in” embodiments. The oscillator assembly 502 may be driven by a motor 506. The motor 506 may include a drift shaft that rotates during operation. The drive shaft may be coupled to a cam assembly 508. The top of the cam assembly 508 may include a cam. The cam may couple to a proximal end of the shaft 510.

The cam may be configured to, when engaged with the shaft 510, drive the shaft 510 in the lateral direction when the drive shaft rotates the cam assembly 508. In some embodiments, the oscillator assembly 502 may include a sleeve 512 that houses a section of the shaft 510. In an exemplary embodiment, the cam may comprise a projection that is received in a pocket of the sleeve 512. As the cam rotates, the projection causes the sleeve 512 to move laterally following the path of the cam rotation. The shaft 510 moves laterally with the sleeve 512. In some embodiments, the handle 400 may include one or more guide plates 516. The guide plates 516 may include one or more slots through which the shaft 510 is able to pass through. The slots may include a width that defines a range of movement that limits the travel of the shaft 510 in the lateral direction. The guide plates 516 ensure that the shaft 510 stays within a restricted path of motion. Some embodiments may include a cover 514 that prevents water from entering the handle and contacting the internal elements.

Generally, the signal is processed and then analyzed by the teeth cleaning system (e.g., teeth cleaning system 100 of FIG. 1). For example, proprietary machine learning could be employed “in device” such that the health of a user is locally analyzed without requiring that the signal be transmitted external to the teeth cleaning system. In such embodiments, analyses of the signal may be transmitted external to the teeth cleaning system, for example, to a computer program executing on an electronic device that is communicatively connectable to the teeth cleaning system. Through the computer program, a user may be able to review the analyses. Note that the “user” of the computer program could be the person whose teeth are brushed by the teeth cleaning system or some other person.

Alternatively, the signal could be processed and/or analyzed external to the teeth cleaning system. For example, the signal may be forwarded—in its raw form or processed form—to an electronic device for analysis by a computer program executing thereon. The signal could be streamed to the electronic device in near real time as its values are generated, or the signal could be transmitted to the electronic device such that values are received in “batches.” For example, the values may be forwarded by the teeth cleaning system to the electronic device when brushing ends, when the teeth cleaning system is recharged, when a predetermined time (e.g., 12 AM) has passed, or when a predetermined duration (e.g., 24 hours) has elapsed. Accordingly, values could be provided to the electronic device for analysis by the computer program in a continual, periodic, or ad hoc manner.

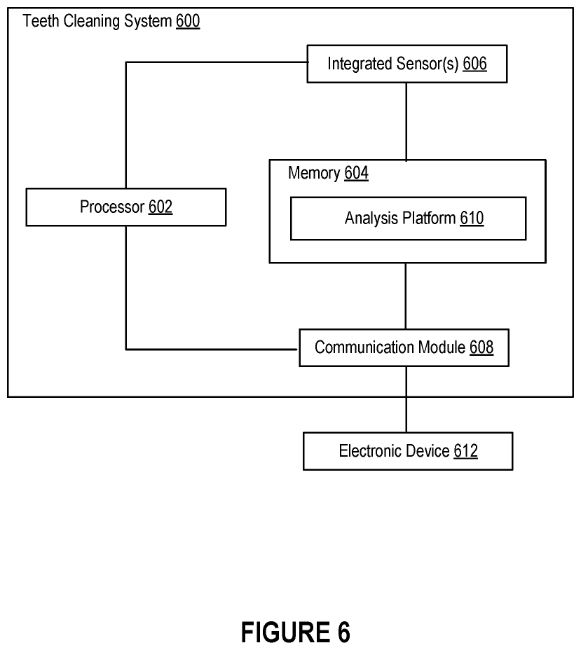

FIG. 6 illustrates an example of a teeth cleaning system 600 that is able to implement a computer program—called an “analysis platform” 610—designed to parse signals generated by integrated sensors of the teeth cleaning system. As shown in FIG. 6, the teeth cleaning system 600 can include a processor 602, memory 204, integrated sensors 206 and communication module 208. Each of these components is discussed in greater detail below. Those skilled in the art will recognize that other components may be present. For example, the teeth cleaning system 600 may include an oscillator assembly as discussed above with reference to FIGS. 4-5.

The processor 602 can have generic characteristics similar to general-purpose processors, or the processor 602 may be an ASIC that provides control functions to the teeth cleaning system 600. The processor 602 can be coupled to all components of the teeth cleaning system 600, either directly or indirectly, for communication purposes.

The memory 604 may be comprised of any suitable type of storage medium, such as static random-access memory (“SRAM”), dynamic random-access memory (“DRAM”), electrically erasable programmable read-only memory (“EEPROM”), flash memory, or registers. In addition to storing instructions that can be executed by the processor 602, the memory 604 can also store data generated by the processor 602 (e.g., when executing the modules of the analysis platform 610). Note that the memory 604 is merely an abstract representation of a storage environment. The memory 604 could be comprised of actual integrated circuits (also called “chips”).

The integrated sensors 606 can monitor for the presence or concentration of analytes in saliva. Generally, the teeth cleaning system 600 either includes a single integrated sensor for measuring a single analyte or multiple integrated sensors for measuring multiple analytes. However, the teeth cleaning system 600 could include multiple integrated sensors for measuring the same analyte, for example, for verification purposes. In operation, each integrated sensor can produce, as output, a signal that is indicative of the presence or concentration of a corresponding analyte. Generally, the signal is representative of a sequence of values in temporal order that indicate the presence or concentration of the corresponding analyte at corresponding points in time. The signals generated by the integrated sensors 606 may be provided to the analysis platform 610 for processing and/or analyzing.

While the integrated sensors 606 should ideally be able to produce signals as output as expeditiously as possible, several minutes (e.g., 2-10 minutes, and preferably 20 seconds to 3 minutes) may be acceptable as that is roughly the amount of time that a user would be expected to keep the teeth cleaning system 600 in her mouth. Similarly, while durability would ideally be months or years, durability of several weeks (e.g., at least 2-3 weeks) may be acceptable.

The communication module 608 may be responsible for managing communications external to the teeth cleaning system 600. The communication module 608 may be wireless communication circuitry that is able to establish wireless communication channels with other electronic devices. Examples of wireless communication circuitry include 2.4 gigahertz (GHz) and 5 GHz chipsets compatible with Institute of Electrical and Electronics Engineers (IEEE) 802.11—also referred to as “Wi-Fi chipsets.” Alternatively, the communication module 208 may be representative of a chipset configured for Bluetooth®, Near Field Communication (NFC), and the like.

The nature, number, and type of communication channels established by the teeth cleaning system 600—and more specifically, the communication module 608—may depend on (i) the number of sources from which data is acquired and (ii) the number of destinations to which data is transmitted. Assume, for example, that the analysis platform 610 resides on the teeth cleaning system 600 as shown in FIG. 6. In such embodiments, the teeth cleaning system 600 may communicate with a mobile phone associated with the user and a computer server of a server system that is responsible for supporting the analysis platform 610. In some embodiments, said mobile phone may not only receive data from said teeth cleaning system 600, but may be able to transmit data to said teeth cleaning system 600.

For convenience, the analysis platform 610 is referred to as a computer program that resides in the memory 604. However, the analysis platform 610 could be comprised of hardware or firmware in addition to, or instead of, software. As mentioned above, the analysis platform 610 may process the signals generated by the integrated sensors 606 and then forward the processed signals—or analyses of the processed signals—to a computer program executing on an electronic device 612 for further analysis. In some embodiments, said analysis platform 610 and/or processor 602 may include electrochemical analyzers components to assist in processing and analyzing readings from the integrated sensors 606. The computer program may be representative of another instance of the analysis platform 610, or the computer program may be representative of a complementary program to the analysis platform 610. Alternatively, the signals may be forwarded to the communication module 608 in their raw form for transmission to the computer program executing on the electronic device 612. Accordingly, the analysis platform 610 could reside on the teeth cleaning system 600, on the electronic device 612, or both. FIG. 6 includes an embodiment in which the analysis platform 610 resides on an electronic device 612 that is communicatively connected to the teeth cleaning system 600. The electronic device 612 could be a consumer electronic device, such as a mobile phone or tablet computer, that is communicatively and/or electrically couplable to the teeth cleaning system 600, or the electronic device 612 could a specialized electronic device, such as a docking station, that is communicatively and/or electrically couplable to the teeth cleaning system 600. As a specific example, the electronic device 612 could be a docking station that provides power to the teeth cleaning system 600 when the teeth cleaning system 600 is appropriately “docked” thereto. When the teeth cleaning system 600 is “docked” to the docking station, the signals generated by the integrated sensors 606—or analyses of the signals—may be uploaded to the docking station. The docking station may examine the signals, or the docketing station may forward the signals (e.g., to a network-accessible server system, mobile phone, tablet computer, etc.) for further analysis.

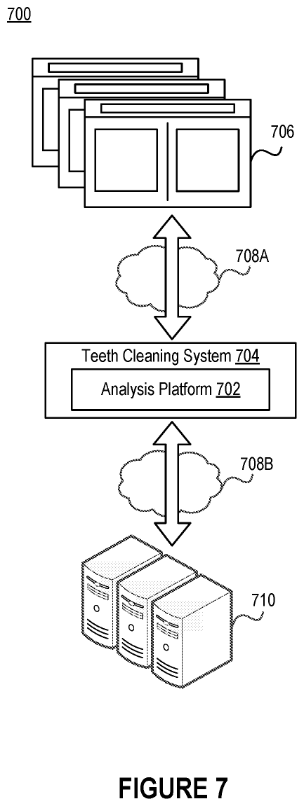

FIG. 7 illustrates a network environment 700 that includes an analysis platform 702 that is executed by a teeth cleaning system 704. Note that in some embodiments, the analysis platform 702 is executed by another electronic device as discussed above. For example, the analysis platform 702 could be executed by a mobile phone or tablet computer that is communicatively connectable to the teeth cleaning system. An individual (also called a “user”) may be able to interact with the analysis platform 702 via interfaces 706. For example, a user may be able to access an interface through which data generated by the integrated sensors—or analyses of that data—is viewable. As another example, a user may be able to access an interface through which insights into the health, or recommendations for improving the health, are viewable. These insights and recommendations may be determined by the analysis platform based on an analysis of the data generated by the integrated sensors.

As shown in FIG. 7, the analysis platform 702 may reside in a network environment 700. Thus, the teeth cleaning system 704 on which the analysis platform 702 resides may be connected to one or more networks 708A-B. Depending on its nature, the teeth cleaning system 704 could be connected to a personal area network (PAN), local area network (LAN), wide area network (WAN), metropolitan area network (MAN), or cellular network. For example, the teeth cleaning system 704 may be communicatively connected to the mobile phone of a user and a computer server of a server system via respective network connections.

The interfaces 706 may be accessible via a web browser, desktop application, mobile application, or another form of computer program. For example, to interact with the analysis platform 702, a user may initiate a web browser on an electronic device and then navigate to a web address associated with the analysis platform 702. As another example, a user may access, via a desktop application, data processed by the analysis platform 702 or insights derived by the analysis platform 702.

As mentioned above, aspects of the analysis platform 702 could be hosted locally, for example, in the form of a computer program executing on the teeth cleaning system 704. Several different versions of computer programs may be available depending on the intended use. Assume, for example, that data generated by the integrated sensors is to be processed locally for security reasons. In such a scenario, the computer program may process the signals and then provide insights derived therefrom to another electronic device for presentation to a user. Alternatively, if the teeth cleaning system is computationally “lightweight,” then signals generated by the integrated sensors may be forwarded to the other electronic device for analysis.

Often, aspects of the analysis platform 702 are distributed across different devices, such as the teeth cleaning system 704, a mobile phone associated with the user, and a computer server that is part of a network-accessible server system 710. As noted above, the teeth cleaning system 704 may be designed to be periodically “docked” to a docking station, for example, for recharging purchases. In embodiments where the teeth cleaning system 704 is accompanied by a docking station, the analysis platform 702 could reside—partially or entirely—on the docking station. Alternatively, the docking station may execute a computer program that, in operation, receives signals from the teeth cleaning system 704 and then forwards the signals to the server system 710 for review.

Thus, the analysis platform 702 may partially or entirely be executed by a cloud computing service operated by, for example, Amazon Web Services®, Google Cloud Platform™, or Microsoft Azure®. In such embodiments, the teeth cleaning system 704 may transmit the signals generated by the integrated sensors to a computer server that is part of a server system 710. Often, the server system 710 is comprised of multiple computer servers. These computer servers can include different types of data (e.g., information regarding users, such as demographic information and health information), algorithms for processing, presenting, and analyzing the signals generated by integrated sensors, and other assets. Those skilled in the art will recognize that this data could also be distributed among the server system 710 and other electronic devices, including the teeth cleaning system 704.

The foregoing description of various embodiments of the technology has been provided for the purposes of illustration and description. It is not intended to be exhaustive or to limit the claimed subject matter to the precise forms disclosed.

Many modifications and variations will be apparent to those skilled in the art. Embodiments were chosen and described in order to best describe the principles of the technology and its practical applications, thereby enabling others skilled in the relevant art to understand the claimed subject matter, the various embodiments, and the various modifications that are suited to the particular uses contemplated.

What is claimed is:

1. A teeth cleaning system for monitoring analytes in saliva comprising:
a handle assembly that is connected to a tray that is configured to be inserted into a user's mouth to surround a user's upper and lower teeth;
said tray comprising at least one aperture that is configured to house at least one integrated sensor;
said integrated sensor that is configured to identify a concentration of an analyte in said user's saliva; and
a transmitter that is configured to transmit a signal that represents said concentration of said analyte in said user's saliva to an external processor;
wherein said integrated sensor is configured to be inserted and removed from said aperture by said user.

2. The teeth cleaning system of claim 1, wherein said teeth cleaning system further comprises said transmitter for transmitting data related to a first concentration and a second concentration to an external network.

3. The teeth cleaning system of claim 2, wherein said external network is configured to be in communications with an app of a user's mobile device.

4. The teeth cleaning system of claim 1, wherein said teeth cleaning system further comprises a receiver for receiving data related to recommendations for said user in response to said concentration from an app of a user's mobile device.

5. The teeth cleaning system of claim 1, wherein at least one electrochemical analyzer is configured to analyze said signal from said at least one integrated sensor.

6. The teeth cleaning system of claim 5, wherein said electrochemical analyzer is configured to be housed in said handle assembly.

7. The teeth cleaning system of claim 1, wherein said at least one integrated sensor is adjacent to said inner wall of said tray, wherein said inner wall of said tray is configured to collect said user's saliva.

8. The teeth cleaning system of claim 7, wherein said at least one integrated sensor further comprises at least two integrated sensors that are adjacent to opposite sides of said inner wall of said tray.

9. The teeth cleaning system of claim 1, wherein said teeth cleaning system further comprises at least a second integrated sensor that is configured to identify a second concentration of a second analyte in said user's saliva.

10. A system for cleaning teeth comprising:
a handle assembly that is connected to a tray that is configured to be inserted into a user's mouth to surround a user's upper and lower teeth;
said tray comprising at least one integrated sensor that is configured to identify a concentration of an analyte in said user's saliva;
an electrochemical analyzer that is connected to said at least one integrated sensor, wherein said electrochemical analyzer is configured to transmit a first signal representing said concentration to a processor; and
a transmitter that is configured to transmit a second signal received from said processor that represents said concentration to an external network.

11. The teeth cleaning system of claim 10, wherein said external network is connected to an app on a mobile device of said user.

12. The teeth cleaning system of claim 10, wherein said integrated sensor is adjacent to an inner wall of said tray, wherein said inner wall of said tray is configured to collect said user's saliva.

13. The teeth cleaning system of claim 12, wherein said integrated sensor further comprises at least two integrated sensors that are adjacent to opposite sides of said inner wall of said tray.

14. The teeth cleaning system of claim 13, wherein said system for cleaning teeth further comprises at least a second integrated sensor that is configured to identify a second concentration of a second analyte in said user's saliva.

15. The teeth cleaning system of claim 10, wherein said external network is configured to be in communication with an app of a user's mobile device.

16. The teeth cleaning system of claim 10, wherein said electrochemical analyzer is configured to be housed in said handle assembly.

17. A teeth cleaning system for analyzing analytes in a user's saliva comprising:
a handle assembly that is connected to a tray that is configured to be inserted into a user's mouth to surround a user's upper and lower teeth;
said tray comprising at least one integrated sensor that is configured to identify a concentration of an analyte in said user's saliva;
a processor that is configured to receive a first signal related to said concentration of said analyte in said user's saliva; and
a transmitter that is configured to transmit a second signal from said processor related to said concentration of said analyte in said user's saliva to an external network;
wherein said integrated sensor is adjacent to an inner wall of said tray and said processor is housed in said handle assembly.

18. The teeth cleaning system of claim 17, wherein at least one integrated sensor further comprises at least two integrated sensors that are adjacent to opposite sides of said inner wall of said tray.

19. The teeth cleaning system of claim 17, wherein said external network is connected to an app on a mobile device of said user.

20. The teeth cleaning system of claim 17, said teeth cleaning system further comprises a receiver for receiving data related to recommendations for said user in response to said concentration from an app of a user's mobile device.
